(19) United States
(12) Patent Application Publication
(10) Pub. No.: US 2023/0181131 A1
(43) Pub. Date: Jun. 15, 2023

(54) SYSTEM AND METHOD FOR MITIGATING RADIATION EXPOSURE

(71) Applicant: Weightless Lead, LLC, New York, NY (US)

(72) Inventor: Babak Hassid, New York, NY (US)

(73) Assignee: Weightless Lead, LLC, New York, NY (US)

(21) Appl. No.: 18/080,270

(22) Filed: Dec. 13, 2022

Related U.S. Application Data
(60) Provisional application No. 63/288,975, filed on Dec. 13, 2021.

(51) Int. Cl. A61B 6/10

(52) U.S. Cl. CPC A61B 6/107

(57) ABSTRACT

The present disclosure relates to a system for protecting surgeons, medical staff and other individuals from radiation and other forms of radiographic exposure. The system includes a stand positioned away from a patient operating table and comprising at least one shielding panel. The system may comprise another shielding panel that slides out from the at least one shielding panel. The system is configured to permit the free movement of a surgeon and medical staff without requiring any individual to wear a lead apron, body armor or zero-gravity suit to protect from radiating devices or equipment in the operating room.

### CROSS-REFERENCE TO RELATED APPLICATIONS

This patent application claims priority, pursuant to 35 U.S.C. § 119(e), to U.S. Provisional Patent Application No. 63/288,975 filed on Dec. 13, 2021, the entire contents of which are hereby incorporated by reference herein.

### COPYRIGHT NOTICE

This disclosure is protected under United States and/or International Copyright Laws. © 2022 Weightless Lead LLC. All Rights Reserved. A portion of the disclosure of this patent document contains material which is subject to copyright protection. The copyright owner has no objection to the facsimile reproduction, for non-commercial use, of this patent disclosure as it appears in the Patent and/or Trademark Office patent file or records, but otherwise reserves all copyrights whatsoever.

### FIELD OF THE INVENTION

This application relates to the fields of Interventional Cardiology, Interventional Neurology/Neurosurgery and Electrophysiology. More specifically, this application relates to systems and methods for reducing exposure to radiographic devices, such as may be introduced during use of an x-ray machine or during the practice of Interventional Radiology.

### BACKGROUND OF THE INVENTION

There are currently different methods available to protect medical professionals from constant radiographic and/or radiation exposure. The most common is wearing lead aprons, such as those manufactured by AADCO Medical, Inc., in their line of Ray Shield® aprons and vests. Similar worn shielding, or basically radiation “armor” such as lead skirts, and lead thyroid collars are extremely heavy as compared to standard surgical garments. Many practitioners must wear these heavy garments for long hours during complicated procedures, or on days when they have several procedures scheduled. Wearing heavy radiation armor rapidly fatigues even a physically fit wearer, and with chronic use can cause orthopedic disorders. As a result, practitioners may suffer shoulder, back, knee and hip pain on a daily basis, and over time may suffer permanent damage, require surgery, or even prematurely end a career (for example, via cancer, or cataracts).

Leaded acrylic face shields and leaded acrylic glass shields have also been used, but they protect only a tiny portion of the body and are ineffective in mitigating the effects of prolonged exposure to radiation.

Other systems and methods rely on cumbersome and/or heavy shielding apparatus. Heavy shielding around the patient can be effective in blocking radiation from medical staff. However, the medical staff still need access to the patient's body, so complete shielding is impractical. Furthermore, because the human body is transparent to X-rays (“radiolucent”), X-rays can shine through the patient's body and expose the medical staff. Any surgery carries with it a risk of life-threatening complications that would require the medical staff to have immediate access to the patient's body, and heavy shielding around the patient's and or a practitioner's body are bulky and difficult to move, which can prevent and/or prohibit access by the medical staff to the patient in such a situation.

One attempted partial solution is to make the garments lighter by using thinner layers of metal or mixing the metals with other materials. This reduces the protection level afforded by the garments, and the overall reduction of the weight of the garment is relatively small. One product line announces a reduction in weight of 30%, reducing the weight from 10.5 pounds to 7.5 pounds, and while this may help in the short term, the practitioner is still carrying several additional pounds of gear every day.

While recent improvements in robotics enable noninvasive microsurgical techniques, the site must still be visualized to adequately guide and control the instruments. Visualization can be accomplished by radiological monitoring, such as X-ray monitoring. During the procedure an X-ray generator is positioned on one side of the patient to emit X-rays to the surgical site (this is generally below the patient, although the position of the X-ray generator can be vary). An X-ray intensifier is positioned to receive the emitted X-rays after they have passed through the surgical site, to convey image data to a monitor to present a visual image to the surgeon/or operator. The x-ray intensifier and generator move around the patient to capture different angles. Unfortunately, the constant radiological monitoring exposes everyone involved to more radiation than was required using the old techniques. While such exposure is a minor concern for the patient, the professional medical staff who perform these procedures have much more frequent exposure, and the cumulative exposure exceeds safe limits.

Another solution is zero gravity radiation protection systems. “Zero gravity” suits are leaded body suits that are suspended by a rigid frame mounted on the floor or ceiling so the wearer does not support the suit with his or her body. The device is very large and not user friendly. Such armor leaves the wearer's hands and lower arms uncovered and unprotected and limits the wearer's range of bodily movement to movements that can be accommodated by the frame. The draping of the device takes a very long time and the learning curve to use the device makes many practitioners to not want to use it. The device prevents the natural movement needed in the operating room, and although there is no significant weight, there is a constant feeling of pressure on the practitioner making it uncomfortable and not user friendly. Such armor systems are also extremely expensive due to their complexity and due to material costs.

Another form of radiation armor is the mobile “cabin,” that is a radiopaque box on wheels in which the user stands. The user can push the cabin from place to place while inside. The cabin has arm ports at a certain height and a visually transparent portion at a certain height. The user's hands and face cannot be repositioned or reoriented much, for example to stand or lean over. It also uses a static face shield that prevents the wearer from bringing anything close to the face, for example for visual scrutiny.

More recently, other prior art has attempted to address these issues by offering a rotatable shielding solution while creating other problems. For example, rotatable shielding devices interferes with the motion of the X-ray camera or cameras and other equipment during certain procedures. Another problem is the device's interaction with the catherization lab equipment including a camera, x ray intensifier and operating room table. These devices are placed under the operating table and every time the camera moves, it hits it, which over time causes collateral damage to the camera/image intensifier (often exceeding $1 M in cost). Because it is positioned under the operating table and because that camera head is always moving, and because practitioners need specific angles of the camera to come down, this close proximity prevents the operator from getting the full images of the patient, and limits the angles needed to treat a patient. Because it needs to go under the table, the lead apron shield on the table cannot go all the way up to the table, thereby presenting the potential for the radiation to get through and affect those on the right side of the device. As a result, operators opt to wear lead protection because of the risk that the lead will affect their gonads or other vulnerable organs. Finally, it has a very cumbersome setup. It requires three panels and is therefore very cumbersome to get it draped for preserving sterility.

There is therefore a long-felt and unresolved need to shield medical staff from X-rays to which a patient must be exposed that does not encumber the user's body, allows access to the patient's body, can be rapidly reconfigured if necessary, and does not interfere with the motion or positioning of X-ray cameras or other equipment during medical procedures. Other advantages over the prior art will become known upon review of the Summary, Detailed Description and the appended claims.

### SUMMARY OF THE INVENTION

According to one aspect of the present disclosure, a novel system and method is described for eliminating, reducing and/or mitigating exposure from radiographic equipment, such as X-ray and similar radiation-based equipment which a patient and medical staff must utilize while in a medical and/or operating setting. In embodiments, the system and method incorporates at least one shield that is easily manipulated and maneuvered, does not encumber a user or a patient's body, allows access to the patient's body, can be rapidly reconfigured when necessary, and does not interfere with the motion or positioning of X-ray cameras or other equipment during medical procedures.

By way of providing additional background, context, and to further satisfy the written description requirements of 35 U.S.C. § 112, the following are incorporated by reference in their entireties for the express purpose of explaining and further describing surgical procedures and commonly used equipment in such procedures: U.S. Pat. No. 6,309,395 to Smith et al.; U.S. Pat. No. 6,142,998 to Smith et al.; U.S. Pat. No. 7,014,640 to Kemppanien et al.; U.S. Pat. No. 7,406,775 to Funk, et al.; U.S. Pat. No. 7,387,643 to Michelson; U.S. Pat. No. 7,341,590 to Ferree; U.S. Pat. No. 7,288,093 to Michelson; U.S. Pat. No. 7,207,992 to Ritland; U.S. Pat. No. 7,077,864 Byrd III, et al.; U.S. Pat. No. 7,025,769 to Ferree; U.S. Pat. No. 6,719,795 to Cornwall, et al.; U.S. Pat. No. 6,364,880 to Michelson; U.S. Pat. No. 6,328,738 to Suddaby; U.S. Pat. No. 6,290,724 to Marino; U.S. Pat. No. 6,113,602 to Sand; U.S. Pat. No. 6,030,401 to Marino; U.S. Pat. No. 5,865,846 to Bryan, et al.; U.S. Pat. No. 5,569,246 to Ojima, et al.; U.S. Pat. No. 5,527,312 to Ray; and U.S. Pat. Appl. No. 2008/0255564 to Michelson.

Accordingly, one aspect of the present application relates to a system, which in a preferred embodiment comprises, but is not limited to: a stand positioned away from a patient operating table; at least one shielding panel; another shielding panel that slides out from the at least one shielding panel; wherein the system is configured to permit the free movement of a surgeon and medical staff without requiring any individual to wear a lead apron, body armor or zero-gravity suit to protect from radiating devices or equipment in the operating room.

According to another aspect, the present application relates to a method for protecting an individual from exposure to radiation occurring from radiographic equipment in an operating room, comprising the steps of: positioning a stand supporting an arm and at least one shielding panel away from an operating table; extending the stand to a desired height for positioning the at least one shielding panel above the operating table; positioning the at least one shielding panel between the individual and the radiographic equipment; and removing the stand and at least one shielding panel from the operating table when the radiographic equipment is no longer in use.

According to yet another aspect, the method further comprises at least a second shielding panel that is coupled to the at least one shielding panel, wherein the method further comprises the step of: extending the at least a second shielding panel away from the at least one shielding panel and increase the shielding area.

According to yet another aspect, the system and method comprise at least a second shielding panel coupled to at least one shielding panel by a telescoping engagement.

According to yet another aspect, the method further comprises the step of retracting the at least a second shielding panel prior to the step of removing the stand.

According to yet another aspect, the method further comprises the step of securing the stand in the desired height by locking means.

According to yet another aspect, the at least one shielding panel of the system is transparent and comprised of lead-based glass material. In yet another aspect, the at least one shielding panel is opaque and comprised of a lead-based acrylic material.

According to yet another aspect, the at least one shielding panel comprises a skirt extending from a lower surface of the at least one shielding panel.

According to yet another aspect, the stand comprises a plurality of rollers for positioning the stand away from the operating table.

According to yet another aspect, the arm does not extend longer than the length of the at least one shielding panel.

According to yet another aspect, the shielding panels are not permitted to rotate relative to each other or to the stand.

The Summary is neither intended nor should it be construed as being representative of the full extent and scope of the present disclosure. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

The above-described benefits, embodiments, and/or characterizations are not necessarily complete or exhaustive, and in particular, as to the patentable subject matter disclosed herein. Other benefits, embodiments, and/or characterizations of the present disclosure are possible utilizing, alone or in combination, as set forth above and/or described in the accompanying figures and/or in the description herein below. However, the claims set forth herein below define the invention.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

In the drawings:

FIG. 1A is a perspective view of various components of the system according to embodiments of the present disclosure;

FIG. 1B is another perspective view of the system illustrated in FIG. 1A;

FIG. 1C is another perspective view of the system illustrated in FIG. 1A;

FIG. 2 is a perspective view of the system according to another embodiment of the present disclosure; and

FIG. 3 is a side elevation view of the system according to another embodiment of the present disclosure.

### DETAILED DESCRIPTION

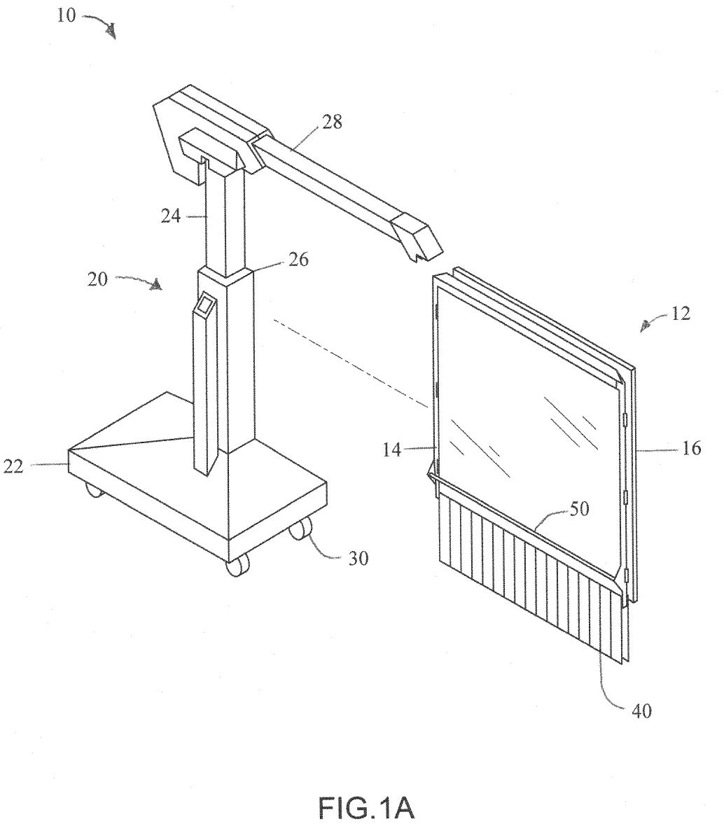

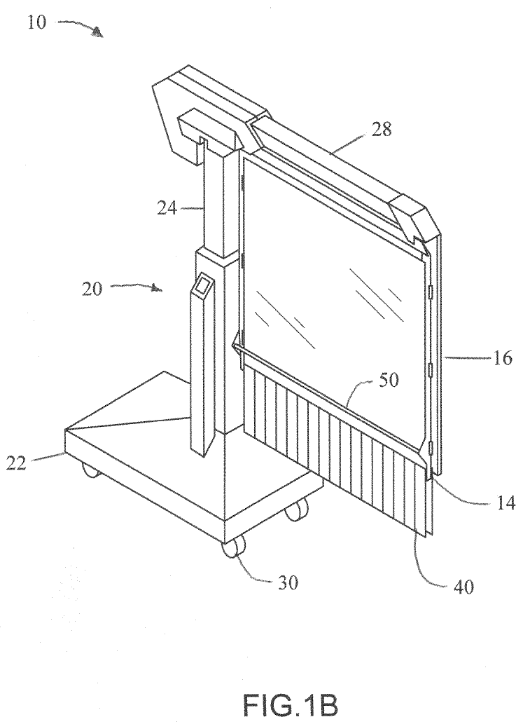

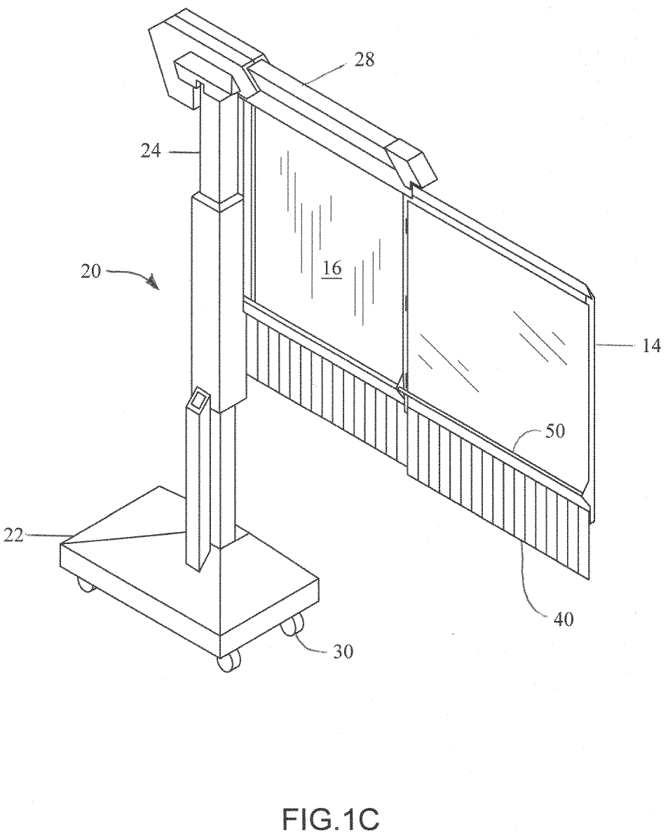

As shown in FIGS. 1-3 and described in further detail herein, the present disclosure relates to a novel system and method for reducing, mitigating and in some instances eliminating exposure to radiation and/or radiographic equipment during a variety of surgical procedures. Reference is now made to FIGS. 1A-1C, which illustrates one preferred embodiment of the present disclosure. As shown in FIGS. 1A and 1n varying embodiments, the system 10 comprises at least one shield 12. The shield 12 may consist of two panels 14, 16, which preferably are comprised of a transparent or semi-transparent glass lead material. Alternatively, one or more of the panels 14, 16 may be comprised of lead or equivalent shielding material and is non-transparent. More preferably, the system 10 is comprised of a first panel 14 comprised of a glass lead transparent material, and a second panel 16 that is attached to and selectively extendable from the first panel and is comprised of a lead or lead-equivalent material and is non-transparent.

As shown in FIGS. 1A-1C, the system 10 comprises a stand 20. According to embodiments, the stand 20 supports both the first panel 14 and second panel 16 while permitting the base 22 of the stand 20 to be positioned away from the patient while simultaneously avoiding contact with the medical staff. The stand 20 preferably supports at least one stationary lead shielding or second panel 16 and another shielding panel (or first panel) 14 that is configured to extend outwardly from the area shielded by the second panel 16 to extend coverage (i.e., shielding) an area of particular focus during a specific surgical procedure. By way of example but not limitation, a particular surgical procedure may be focused on a patient's abdomen, and the first panel 14 may be positioned to extend above (or below) the patient's abdomen and thereby shield from radiation to an individual(s) present during the surgery who is operating on the patient. The position of the first panel 14 is in addition to the position of the second panel 16, which may be more appropriately positioned to shield an individual(s) from radiation sources in the operating room. The positioning of the first and second panels 14, 16 in a preferred embodiment is sufficient to mitigate risk of exposure to radiation and other radiographic exposure such that the individual(s) no longer need to wear a lead apron or other shielding apparatus. The need for lead aprons is eliminated in part because of the configuration and the positioning of the device in relation to the patient and the doctors/technicians.

The stand 20 supports the first and second panels 14, 16 in a configuration that permits the panels 14, 16 to be adjusted in height relative to the floor surface supporting the stand 20. In a preferred embodiment, the stand 20 comprises a vertical support element 24 that comprises a track or slide 26, wherein the track or slide 26 allows the vertical support element 24 to be raised or lowered relative to the base 22 of the stand 20. In a most preferred embodiment, the stand also comprises a substantially horizontal arm 28 that is attached to the vertical support element 24 such that the first and second panels 14, 16 are suspended from the arm 28.

In use, the system 10 may be adjusted in height to accommodate the preferred height of the operating table, the size and position of the patient, the preferences of the surgeon or other medical staff, the location of equipment located in the operating room (for example, cameras or other equipment) mounted to the floor, walls or ceiling of the operating room, and the position of any radiation-emitting sources, such as an x-ray machine.

Referring to FIG. 1C, the system 10 is shown with the first and second panels 14, 16 in an extended position. In embodiments, the first panel 14 is telescopically coupled to the frame of the second panel 16 such that the first panel 14 may be extended in a variety of ways relative to the position of the stand 20. This arrangement is preferable because the equipment, operating table, position of the patient, etc. are different from one procedure to the next procedure. In a preferred embodiment the first panel 14 is comprised of a lead glass material such that a surgeon or other medical staff may see through the first panel 14 when it is in an extended position from the second panel 16. The second panel 16 may be comprised of a lead-based material suitable for shielding, for example, a lead-acrylic paneling material. As shown in FIGS. 1A-1C, one or more panels 14, 16 may further comprise a skirt 40 that is preferably comprised of a flexible lead-based material. The skirt 40 may comprise multiple slits such that the associated panel may be placed directly above a piece of equipment, the operating table or a portion of the patient's anatomy without creating gaps in the shielding. In other words, the skirt 40 is comprised of a material that is flexible to substantially conform to the contour of the underlying surfaces, whether those surfaces are needed to be moved during the operation or not.

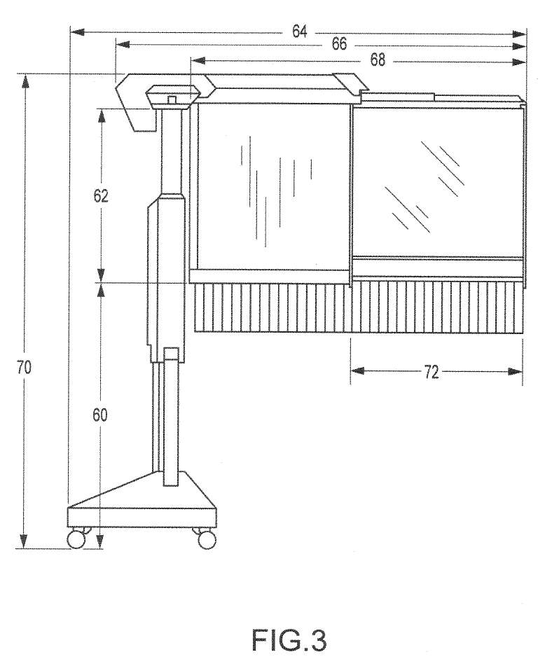

In embodiments, the width of the first and second panels 14, 16 is preferably in the range of ¼ inch to 2 inches, and more preferably in the range of ½ inch to 1½ inches. In a most preferred embodiment the panels 14, 16 are approximately 1 inch wide. Referring to FIG. 3, the panels 14, 16 are preferably large enough to shield an area of approximately 68-76 inches long by 30-42 inches tall (in a fully extended position. Each panel may be approximately 35-38 inches square, although other shapes and sizes are expressly contemplated in this disclosure. One or more of the panels 14, 16 may comprise a handle 50 on a lateral side of the panel for positioning the panels 14, 16 in a retracted or telescopically extended position, as well as desired positions therebetween.

The entire system 10 may be selectively positioned in several different configurations. In certain embodiments, the position of the base 22, the height of the stand 20, and the locations of the first panel 14 and second panel 16 may be set to preference by the user and secured or locked into place to avoid undesired movement. In a preferred embodiment the panels 14, 16 do not rotate or sway due to the telescopic coupling between the two panels. In a preferred embodiment the entire assembly weighs less than 350 lbs., and in a most preferred embodiment the assembly weighs less than 300 lbs.

In embodiments, and referring now to FIG. 1A, panels may be substituted as desired by the user. For example, a first panel 14 may be replaced by another first panel 14 of a different configuration or material. The same substitution may be made for the second panel 16. In certain instances the surgeon may desire to have the first and second panels 14, 16 swapped to provide a viewable panel in one position as opposed to another. In certain instances the user may wish to have both panels 14, 16 opaque, whereas in other instances the user may wish to have both panels 14, 16 transparent.

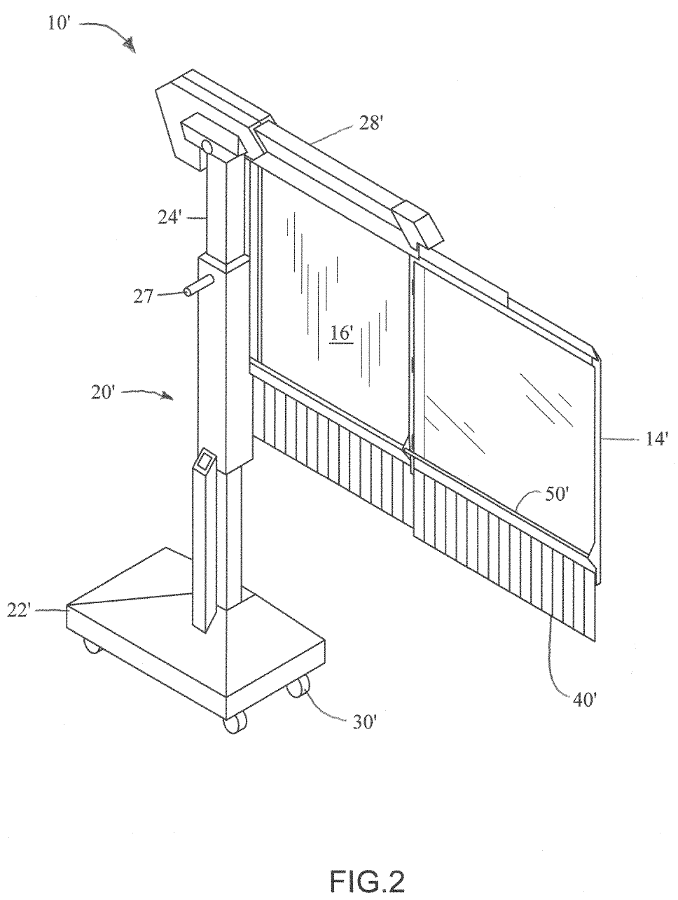

An alternative embodiment of the present disclosure is illustrated in FIG. 2. As described above, the system 10 may comprise a first and second panel 14′, 16′ with the first panel 14′ in a preferably telescoping relationship to the second panel 16′ as depicted. The panels may be comprised of different materials, including lead-based acrylic or other shielding material suitable for preventing and/or mitigating transmission of radiation or other radiographic emission. The stand 20′ may comprise a grip 27 that assists a user in raising or lowering the height of the system 10. While the stand 20′ is coupled to a base 22′ that is permitted to rotate (for example, by the presence of multiple rollers or casters 30′) the vertical support element 24′ preferably does not rotate. The arm 28′ preferably supports one or more panels 14′, 16′ while permitting one of the panels 14′ to extend telescopically from the other panel 16′ as illustrated in FIG. 2. The panels may comprise a skirt 50′ as shown in the embodiments of FIGS. 1A-1C.

Additional aspects of the embodiment of FIG. 2 are shown in the side elevation view of FIG. 3, including preferred dimensions of the components described in detail above. It is to be expressly understood that variations in these dimensions are contemplated and within the scope of the inventions claimed herein. Referring to FIG. 3, the height 70 of the stand 20 in a first or lowered position is preferably in the range of 50-92 inches, more preferably in the range of 60-80 inches, and most preferably in the range of 68-75 inches. The base 22 is preferably wide enough to support the weight of the vertical support element 24, arm 28 and first and second panels 14, 16, and in a preferred embodiment is rectangular in shape when viewed in plan. The base 22 may comprise a plurality of rollers or casters 30. In a preferred embodiment the base 22 has a major dimension in the range of 20-44 inches and a minor dimension in the range of 14-26 inches. In a more preferred embodiment, the base 22 has a major dimension in the range of 26-38 inches and a minor dimension in the range of 16-24 inches. In the most preferred embodiment, the base 22 has dimensions of approximately 32 inches in a major dimension and 20 inches in a minor dimension.

The system 10 may be adjusted such that the stand is in a second or extended position, as shown in FIG. 1C. Referring to FIG. 3, the height 70 of the stand 20 in a second or raised position is preferably in the range of 88-112 inches, more preferably in the range of 94-106 inches, and most preferably in the range of 98-104 inches. In a preferred embodiment, the vertical support element 24 may be adjusted in height 62 between the first or lowered position and the second or raised position shown in FIGS. 1B and 1C. Referring again to FIG. 3, this extension of the vertical support element 24 increases the height 60 between the distal end of the base 22 and the lowest surface of the first and second panels 14, 16. Variations on the height, as well as the height adjusting mechanism, are expressly considered part of the present disclosure. By way of example, the height may be adjusted by a clamp or crimp mechanism, by a ratchet and geared track mechanism, by a locking pin, or other securing means known to those of skill in the art.

Still referring to FIG. 3, the length 72 of the first panel 14, when extended from the second panel, may be sufficient to reach a combined length 68 of up to 76 inches. The length of the extended panels and horizontal arm 28 (referred to in FIG. 3 as 66) only increases this dimension by approximately 6 inches, and with the additional length 64 of the base, by approximately 10 inches.

In certain embodiments, a user may employ two or more apparatus, including one on each side of a patient, to get full radiation protection from both sides of the room. In this arrangement, both sides of the operating table and sides of the room is protected from radiation, not just the right side of the room.

The system may be used in conjunction with a lead apron shield attached to the operating table. For example, a shield may be coupled, affixed or placed alongside the operating table to help reduce scatter radiation from affecting the user or other individual(s) in the operating room.

The configuration and positioning of the system 10 also avoids interference with the camera or cameras. In addition to the convenience of not hitting and thereby inadvertently moving the camera, certain procedures require a surgeon (including, but not limited to when performing Interventional Neurosurgery procedures) to use two cameras at the same time (by way of example, using a Biplane camera). In this situation, the mobility and adjustability of the system 10 becomes even more critical to the success of the operation. In one embodiment of the invention, the camera may be positioned away from the operating table, while the second camera is positioned lateral to the table. The system is easily positionable relative to both cameras, such that the stand can slide into position, the height adjusted to the desired elevation and the panels to be extended. In this manner, the system 10 may be used in the interventional neurology field, which treats brain aneurysms, acute stroke intervention, and embolizations of brain (AV malformations). In contrast, prior art shield systems prevent or significantly inhibit the use of a second camera or other equipment and are unsuitable for interventional neurology procedures.

Unlike prior systems, the system 10 may be positioned away from the table where the patient is lying. This allows a camera to freely swing under the table without it hitting the device. Other prior art devices are positioned under the table and often hit the device causing potential damage to the camera or hitting the device and therefore moving it out of position and allowing radiation to come through, thereby potentially reach the operator, doctor, technician, or physician's assistant. Similar concerns occur with prior art systems that are mounted to the operating table or other stationary equipment located in the operating room.

In one embodiment of the present invention, a camera may be situated at least 28 inches away from the operating table, the technician rarely ever needs that type of angulation in a procedure to come close to the base of the equipment. Because of its position away from the table, a second biplane camera can be used in certain procedures where it will not interfere. Because the preferred embodiment of the present invention is at least 28 inches away, the skirt 50 goes all the way up toward the patient's head and protects them from radiation exposure.

In embodiments, the base, stand, arm and panels described herein are all configured to receive a sterile drape or other covering as required for the procedure. In a preferred embodiment, the system 10 uses a two-step technique to permit installation of a sterile drape up and in position, which unlike prior art systems allows the drape to be installed within minutes. As a result, with the present invention, there is less of a hindrance to drape or to use the system 10.

Previous devices are only configured to move in certain directions. In one embodiment, the arm is attached to the vertical support element and moves as a unit together, as opposed to one shield moving in one direction and another shield in another direction. This allows for easier setup and storage of the device, as well as prohibiting unwanted rotation or movement of the individual shields. In yet another embodiment of the invention, one shield is stationary and the other shield slides out for easier positioning on the patient abdomen, making the device easier for set up and storage. In yet another embodiment of the invention, one shield is vertically hung, and 90 degrees on the patient's abdomen and protrudes out to, for example, 3 feet, in order to block radiation.

The invention features a simple, small footprint, user-friendly device that allows the device to be placed at 90° (i.e., perpendicular) over the abdomen, and, with the use of a standard shield apron that is already in place on the operating table, the radiation is attenuated. In one embodiment of the present invention, two panels slide out at 90° and slide back in after use. The vertical piece comes out from the side, and there is therefore no need for a pole in the middle. In this embodiment, there are only two pieces, and one extends out and over the abdomen for radiation protection. In this way, the system allows 90° access to the patient while limiting the footprint of the apparatus.

With respect to the embodiments described above, it is expressly understood that such embodiments may be incorporated for use in practicing the novel methods described herein. In certain embodiments, those methods may comprise greater or fewer steps than as described below. In use, the portable base which is placed into position and locked and the shield is protruded outwards for radiation protection. In a preferred step, two panels slide out at 90° wherein a first shield extends out from the side of the stand, and a second shield extends from the end of the first shield. There is therefore no need for a pole in the middle. In this embodiment, there are only two pieces, and one extends out and over the abdomen for radiation protection. In this way, the present invention allows 90° access to the patient.

Unlike all prior systems, the base is far away from the camera in the operating room, allowing full motion of the camera to take the different views needed without hitting the device. Steep angles are needed for complex procedures and embodiments of this invention enable the user to obtain these images. During imaging procedures, there is a need for a camera on top of the patient, and an image intensifier underneath the camera. Robotic arm, one goes right, one goes left, one goes under the patient, one goes over, depending on which way the operator moves it. All such movement is permitted and possible with the preferred embodiments of the present invention in a way that was not previously possible with any prior systems.

With the shield in position, the operator and the whole team at the procedure are protected from the radiation with full range of motion and without any inhibition. The shield of the preferred embodiment of the invention protects the whole body, head, eyes, and upper torso, which standard lead aprons do not. This is especially important for medical professionals who work with radiation, having a higher rate of radiation induced cataracts and left-sided brain cancers as compared to the general population.

In yet another embodiment of the invention, certain procedures need the use of two cameras, which is, in one embodiment, called biplane angiography. With the position of this device away from the operating table, the use of the second camera is possible. This is especially important for neuro-surgical procedures, such as, for example, coiling, stenting or acute stroke cases.

In another embodiment of the invention, the device is attached to the ceiling over the operating table. For example, a track (similar to the arm described in relation to FIGS. 1A-1C above) may be constructed in the ceiling or alternatively mounted to the ceiling, and the first and second panels mounted thereon. The device according to this embodiment may be height adjustable and/or configurable to be elevated up or down, or positioned properly in relation to the patient. In this embodiment, the second panel may slide outwardly from the first panel and lock into place, which allows for full range of motion of the image intensifier/camera to allow all different steep angles to be taken while also allowing staff the benefit of radiation protection.

One having skill in the art will appreciate that embodiments incorporating positioning and extending of shields, as well as other embodiments discussed herein, may be used in conjunction with devices that employ automated or semi-automated manipulation, such as, for example, robotics or other autonomous systems. Embodiments may also be configured such that the stand and/or shields may be manipulated, in whole or in part, remotely by an operator, remotely by an operator through a computer controller, by an operator using proportioning devices, programmatically by a computer controller, by servo-controlled mechanisms, by hydraulically-driven mechanisms, by pneumatically-driven mechanisms or by piezoelectric actuators. These apparatus and systems may be programmed to operate with the claimed invention by automated or semi-automated means.

One having skill in the art will appreciate that embodiments of the present disclosure may have various sizes. The sizes of the various elements of embodiments of the present disclosure may be sized based on various factors including, for example, the anatomy of the patient, the location and/or position of the person(s) operating with or otherwise using the system, the surgical site location, physical features of the equipment, devices and instruments used with the systems described herein, including, for example, width, length and thickness, and the size of the surgical equipment utilized during an operation.

Embodiments of the present disclosure present several advantages over the prior art including, for example, enhanced speed and efficacy of positioning and manipulating the components of the system and thereby the surgical procedure, the ability to further reduce or mitigate the effects of radiation, the optimal positioning of radiographic equipment and related surgical equipment, the enhanced ability of medical staff to be adjacent the surgical site with minimal risk of exposure, the improved ability to reposition the components of the system as needed during changes in the procedure, the larger area protected by the positioning of the stand and/or shields described herein, and the ease of manipulation of the system in general.

One having skill in the art will appreciate that embodiments of the present disclosure may be constructed of materials known to provide, or predictably manufactured to provide the various aspects of the present disclosure. These materials may include, for example, stainless steel, titanium alloy, aluminum alloy, chromium alloy, and other metals or metal alloys. These materials may also include, for example, PEEK, carbon fiber, ABS plastic, polyurethane, polyethylene, photo-polymers, ployamide, resins, particularly fiber-encased resinous materials rubber, nylon, latex, synthetic rubber, synthetic materials, polymers, and natural materials.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. For further illustration, the information and materials supplied with the provisional application from which this application claims priority are expressly made a part of this disclosure and incorporated by reference herein in their entirety.

It is expressly understood that where the term “patient” has been used to describe the various embodiments of the disclosure, the term should not be construed as limiting in any way. For instance, a patient could be either a human patient or an animal patient, and the apparatus and methods described herein apply equally to veterinary science as they would to surgical procedures performed on human anatomy. The systems and methods described herein therefore have application beyond surgical procedures specifically referenced herein, and the concepts may be applied to other types of “patients” and procedures without departing from the spirit of the present disclosure.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

The present inventions, in various embodiments, include components, methods, processes, systems and/or apparatuses substantially as depicted and described herein, including various embodiments, subcombinations, and subsets thereof. Those of skill in the art will understand how to make and use the present inventions after understanding the present disclosure. The present inventions, in various embodiments, include providing devices and processes in the absence of items not depicted and/or described herein or in various embodiments hereof, including in the absence of such items as may have been used in previous devices or processes, e.g., for improving performance, achieving ease and\ or reducing cost of implementation.

This application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms. In addition, the headings in this application are for reference purposes only and shall not in any way affect the meaning or interpretation of the present invention.

Although the foregoing text sets forth a detailed description of numerous different embodiments, it should be understood that the scope of protection is defined by the words of the claims to follow. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

What is claimed is:

1. A method for protecting an individual from exposure to radiation occurring from radiographic equipment in an operating room, comprising:
positioning a stand supporting an arm and at least one shielding panel away from an operating table;
extending the stand to a desired height for positioning the at least one shielding panel above the operating table;
positioning the at least one shielding panel between the individual and the radiographic equipment; and
removing the stand and at least one shielding panel from the operating table when the radiographic equipment is no longer in use.

2. The method of claim 1, further comprising at least a second shielding panel that is coupled to the at least one shielding panel, wherein the method further comprises the step of:
extending the at least a second shielding panel away from the at least one shielding panel and increase the shielding area.

3. The method of claim 2, wherein the at least a second shielding panel is coupled to the at least one shielding panel by a telescoping engagement.

4. The method of claim 3, further comprising the step of retracting the at least a second shielding panel prior to the step of removing the stand.

5. The method of claim 4, further comprising the step of securing the stand in the desired height by locking means.

6. The method of claim 1 wherein the at least one shielding panel is transparent and comprised of lead-based glass material.

7. The method of claim 1 wherein the at least one shielding panel is opaque and comprised of a lead-based acrylic material.

8. The method of claim 1 wherein the at least one shielding panel comprises a skirt extending from a lower surface of the at least one shielding panel.

9. The method of claim 1 wherein the stand comprises a plurality of rollers for positioning the stand away from the operating table.

10. The method of claim 1 wherein the arm does not extend longer than the length of the at least one shielding panel.

11. The method of claim 2 wherein the at least one shielding panel and the at least a second shielding panel are not permitted to rotate relative to each other or to the stand.
